(19) United States
(12) Patent Application Publication
(10) Pub. No.: US 2025/0334563 A1
(43) Pub. Date: Oct. 30, 2025

(54) SYSTEM AND METHOD FOR ASSESSING PAVEMENT CONDITIONS

(71) Applicant: Roadway Management Technologies, Little Rock, AR (US)

(72) Inventor: Candler McCollum, Little Rock, AR (US)

(73) Assignee: Roadway Management Technologies, Little Rock, AR (US)

(21) Appl. No.: 18/645,191

(22) Filed: Apr. 24, 2024

(51) Int. Cl. G01N 33/38; G01S 7/02; G01S 7/282; G01S 13/88

(52) U.S. Cl. CPC G01N 33/383; G01S 7/025; G01S 7/282; G01S 13/885

(57) ABSTRACT

A system and method includes a sensor platform, processors, and memory storing sensor data collected by the sensor platform. The senor platform may include one or more sensor units and may be mounted to a vehicle. The sensor platform may collect sensor data while the vehicle is driven on pavement. The sensor data may be processed to determine a pavement condition index (PCI) value based on the processed sensor data. The PCI value represents a condition of the pavement and may be used to assess the condition of the pavement relative to a PCI value determined previously by the system using the same or a different sensor platform.

### TECHNOLOGY

The present disclosure to systems and methods for assessing pavement conditions.

### BACKGROUND

Systems for assessing pavement condition of a road network rely on expensive equipment, such as multibeam RADAR, to capture high resolution images of paved roads and assess pavement conditions based on those high resolution images. These systems are expensive to manufacture, operate, and maintain. Such system also require advanced data processing algorithms and more powerful computing resources. Due in part to the above, the data capture of roads may be limited. Thus, decisions with respect to road condition, while based on high resolution images, may be based on road condition a single point in time, rather than continuously over an extended period of time.

### SUMMARY

In one aspect, a system for assessing pavement conditions may include a sensor platform to collect sensor data while at least one vehicle is driving on pavement and a pavement condition generator.

In some embodiments, the sensor platform may include one or more sensor units, the one or more sensor units comprise a RADAR unit, an accelerometer/IMU unit, a camera unit, and a GPS unit. The RADAR unit comprises one or more single-transceiver RADAR sensors. The accelerometer/IMU unit comprises one or more accelerometer/IMU sensors. The camera unit comprises one or more cameras. The GPS unit comprises one or more GPS modules.

In some embodiments, the pavement condition generator may be configured to receive the collected sensor data from the sensor platform, process the collected sensor data to remove outlier data, determine a pavement condition index (PCI) value based on the processed sensor data, and assess a condition of the pavement based on the determined PCI value.

In some embodiments, the RADAR units may emit high-frequency RADAR pulses and low-frequency RADAR pulses directed towards the pavement.

In some embodiments, the system may further include a polarizing filter for polarizing RADAR pulses emitted by the RADAR units in a direction either perpendicular or parallel to a surface of the pavement beneath the vehicle.

In some embodiments, the sensor platform may be configured to simultaneously collect and upload the sensor data to memory while the vehicle is driving on pavement.

In some embodiments, the pavement condition generator may be further configured to determine a PCI value for each sensor unit based on the data collected by that sensor unit.

In some embodiments, the pavement condition generator may be further configured to combine the determined PCI values for each sensor unit into a fused PCI value by applying a sensor fusion algorithm to the determined PCI values for each sensor unit.

In some embodiments, the sensor fusion algorithm may apply a weighted value determined by metadata to each of the determined PCI values for each sensor unit. The metadata may be indicative of a quality of the sensor data collected by each sensor unit.

In some embodiments, the pavement condition generator may be further configured to monitor an accelerometer signal using the accelerometers, detect a perturbance in the accelerometer signal, and initialize the sensor platform based on the detected perturbance.

In some embodiments, the pavement condition generator may be further configured to monitor a distance traveled by the vehicle in a predetermined time period using the GPS module and initialize the sensor platform when the distance traveled by the vehicle exceeds a predetermined distance threshold within the predetermined time period.

In another aspect, a method of assessing pavement conditions may include providing a sensor platform to collect sensor data while at least one vehicle is driving on pavement. The sensor platform may include one or more sensor unit comprising a RADAR unit. The RADAR unit may include one or more single-transceiver RADAR sensors.

The method may further include driving on the pavement; collecting sensor data from the sensor platform while driving the at least one vehicle; transmitting and storing the collected sensor data in memory at a remote location, retrieving the stored sensor data from the memory; processing, using one or more processors, the retrieved sensor data to remove anomalies from the retrieved data; determining, using the one or more processors, a pavement condition index (PCI) value based on the processed sensor data; and assessing, using the one or more processors, a condition of the pavement based on the determined PCI value.

In some embodiments, the one or more sensor units may further include an accelerometer/IMU unit, a camera unit, and a GPS units. The accelerometer/IMU unit may include one or more accelerometer/IMU sensors. The camera unit may include one or more cameras. The GPS unit may include one or more GPS modules

In one example, the RADAR unit may emit high-frequency RADAR pulses and low-frequency RADAR pulses.

In some embodiments, the method may include polarizing, using a polarizing filter, a RADAR pulse emitted by the RADAR unit in a direction either perpendicular or parallel to a surface of the pavement beneath the vehicle.

In some embodiments, the method may include simultaneously collecting, transmitting, and uploading the sensor data to a memory while the vehicle is driving on pavement.

In some embodiments, determining, using the one or more processors, the PCI value based on the processed data may include determining, using the one or more processors, a PCI value for each sensor unit based on the data collected by that sensor unit.

In some embodiments, assessing, using the one or more processors, the condition of the pavement based on the determine PCI value may further include combining, using the one or more processors, the determined PCI value for each sensor unit into a final PCI value by applying a sensor fusion algorithm to the determined PCI values for each sensor unit.

In some embodiments, the sensor fusion algorithm may apply a weighted value determined by metadata to each of the determined PCI values for each sensor unit. The metadata may be indicative of a quality of the sensor data collected by each sensor unit.

In some embodiments, the method may further include monitoring, using the one or more processors, an accelerometer signal; detecting, using the one or more processors, a perturbance in the accelerometer signal; and initializing, using the one or more processors, the sensor platform based on the detected perturbance.

In some embodiments, the method may further include monitoring, using the one or more processors, a distance traveled by the vehicle in a predetermined time period using the GPS sensors; and initializing, using the one or more processors, the sensor platform when the distance traveled by the vehicle exceeds a predetermined distance threshold within the predetermined time period.

In another aspect, a sensor platform may include one or more sensor units configured to collect sensor data while a vehicle is driven on pavement and a pavement condition generator.

The one or more sensor units may comprise one or more of a RADAR unit, an accelerometer/IMU unit, a camera unit, or a GPS unit. A RADAR unit may comprise one or more single-transceiver RADAR sensors. A accelerometer/IMU unit may comprise one or more accelerometer/IMU sensors. A camera unit comprises one or more cameras. A GPS unit may comprise one or more GPS modules.

The pavement condition generator may be configured to receive sensor data collected by the one or more sensor units and determine a pavement condition index (PCI) value for each sensor unit based on the sensor data collected by the sensor unit. The pavement condition generator may be configured to apply a sensor fusion algorithm to the PCI values of at least two sensor units. In one example, the PCI values used may be determined from combined sensor data collected from corresponding sensor units of a plurality of sensor platforms. The fusion algorithm may apply a weighted value to each of the determined PCI values for each sensor unit and combine the weighted PCI values into a fused PCI value. The pavement condition generator may be configured to output or provide an assessment of a condition of the pavement based on the fused PCI value.

In one example, the pavement condition generator may be configured to receive sensor data collected by one or more sensor units of a plurality of sensor platforms. The plurality of sensor platforms may include corresponding of different sensor units. In a further example, the pavement condition generator may be configured to combine the sensor data collected by corresponding sensor units to determine a PCI value for the corresponding sensor units. In a further or another example, the pavement condition generator may be configured to determine a PCI value with respect to a first type of sensor unit using sensor data collected by the first type of sensor unit of one or more sensor platforms and a PCI value with respect to a second type of sensor unit using sensor data collected from the second type of sensor unit of one or more sensor platforms. The pavement condition generator may similarly calculate PCI values with respect to additional types of sensor units. In some embodiments, the one or sensor platforms do not include the same type or combination of types of sensor units.

### BRIEF DESCRIPTION OF THE DRAWINGS

The novel features of the described embodiments are set forth with particularity in the appended claims. The described embodiments, however, both as to organization and manner of operation, may be best understood by reference to the following description, taken in conjunction with the accompanying drawings in which:

FIG. 1A shows a system for assessing a condition of pavement according to various embodiments described herein;

FIG. 1B shows an aspect of the system for assessing a condition of pavement according to various embodiments described herein;

FIG. 2 shows an aspect of the system for assessing a condition of pavement according to various embodiments described herein;

FIG. 3A shows aspects of an example method according to various embodiments described herein;

FIG. 3B shows aspects of an example method according to various embodiments described herein;

FIG. 4A shows aspects of an example method according to various embodiments described herein; and

FIG. 4B shows aspects of an example method according to various embodiments described herein.

### DETAILED DESCRIPTION

The system may utilize a road surveying approach based on collection of low density data at frequent intervals. For instance, the system may employ very frequent collection of large amounts of low density data to monitor road deterioration over time rather than collection of a high resolution single snapshot of a road network at one point in time. This may include various sensors configure to collect road condition data, such as GPS modules, IMUs, RADAR sensors, and cameras. The system may be built utilizing cost conscious materials such that sensor platforms may be mounted and used on multiple vehicles, such as a fleet of municipal owned vehicles. For example, the sensor platform may include IMU sensors and RADAR to collect raw sensor data, other sensors may also be included, such as those described here. RADAR sensors may be positioned to direct energy orthogonal to the plane of the road. For instance, RADAR sensors may be positioned on a vehicle frame. In one embodiment, IMU sensors may be directly or indirectly attached to an axle of the vehicle as to avoid dampening of the sensor signal by the vehicle suspension.

In use, sensor platforms may be mounted on vehicles to passively collect road condition data that may be analyzed to provide information regarding the quality of a road or road network as the vehicles drive about their daily activities. A sensor platform may be configured with a motion trigger power function. For instance, onboard accelerometers may detect perturbations and the sensor platform may be configured to “shake and wake” to power on sensors when a perturbation of preprogramed threshold or signal characteristic, e.g., corresponding to a door opening. The sensor platform may similarly be configured to automatically power off, such as after lack of perturbations over a predetermined threshold over a predetermined period of time. Automated power triggering provides passive data collection operation, e.g., a user may not be required to manually power on the sensor platform for each data collection operation. Automated power triggering may also prevent the sensor platform from draining the vehicle battery. In one embodiment, sensor platforms do not process sensor data for interpretation but rather turn on, collect sensor data, upload sensor data, and turn off. In another embodiment, the sensor platform simultaneously collects and uploads sensor data to a memory.

Sensor data collected by the sensors may be processed by a data processing unit of a pavement condition generator. While sensor platforms may be configured to also process data, in a multiple vehicle collection scheme, it may be more efficient for each sensor platform to collect sensor data and upload the data for combination with sensor data collected by the sensor platform previously, sensor data collected by other sensors platforms, or combination thereof. Thus, in one example, the sensor platforms are configured to collect sensor data and upload the sensor data to a database for combination and processing. In some embodiments, a GPS module of the senor platform may track the distance driven. In one example, collected sensor data is stored locally with respect to the sensor platform and periodically transmitted to a central database for processing along with sensor data collected by other sensor platforms mounted to other vehicles. In one example, the senor data collected from the sensor platforms may be uploaded to the cloud for processing by the data processing unit. In one embodiment, if the GPS falls under a predetermined distance in a certain time period, the sensor platform may be configured to upload the sensor data in its onboard data storage, e.g., via wireless communication protocols and mediums such as cellular, WiFi, or the like.

As described in more detail below, the data processing unit may be configured to weigh the collected sensor data that has been collected over time by recency and then combine the weighed data. This may be performed for each type of sensor data, for example, IMU, RADAR, and camera data. Combining of the data may include determining a Pavement Condition Index (PCI) for each type of sensor data. The system may then be configured to combine the scores as a weighted average with relative weights derived from a variety of metadata.

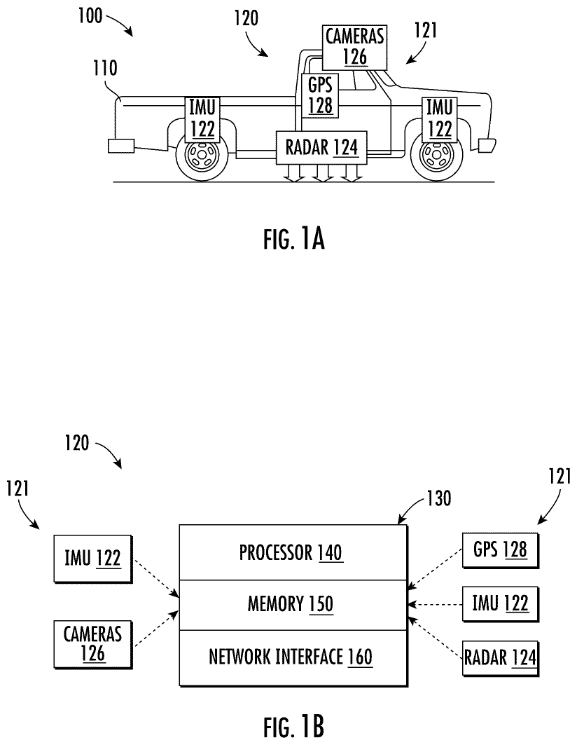

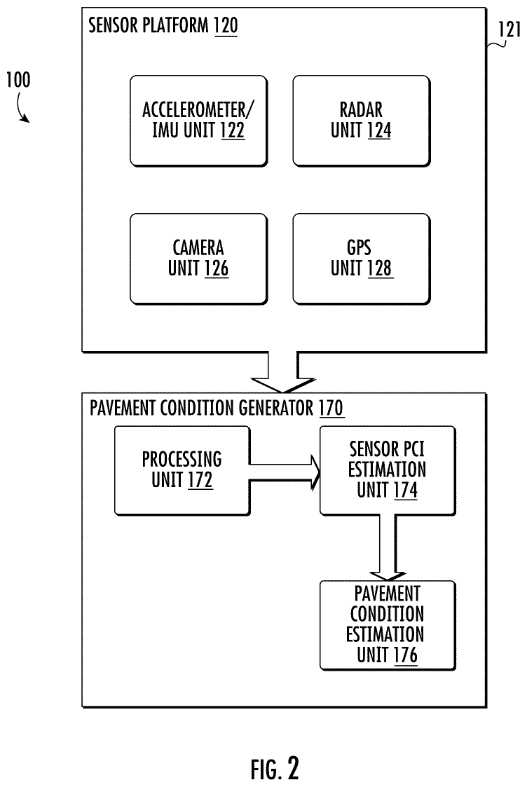

FIGS. 1A and 1B illustrate a system for assessing a condition of pavement according to various embodiments. The system 100 includes or incorporates data collected by at least one sensor platform 120 comprising one or more sensor units 121. The one or more sensor units 121 may include an accelerometer/inertial measurement unit (IMU) 122, a RADAR unit 124, a camera unit 126, and a GPS unit 128. Each sensor unit may include one or more sensors selected from accelerometers/IMU sensors, single-transceiver RADAR sensors, cameras, GPS modules, or combination thereof.

In one embodiment, the accelerometer/IMU units 122 may include one more accelerometers/IMU sensors. The accelerometers/IMU sensors may be attached relative to the axle of the vehicle 110 such that the signal may be received without being dampened by the vehicle's suspension.

The RADAR unit 124 may include one or more RADAR sensors. The RADAR sensors may be positioned to direct energy orthogonal to a plane of the pavement. In one example, RADAR sensors may be mounted to an underside of the vehicle, such as to a frame of the vehicle 110. In various embodiments, RADAR sensors may employ a single transceiver RADAR module. Such modules may be referred to as monostatic radar. Single transceiver RADAR modules may operate by transmitting a pulse of radio waves and then listening for a return signal echo. Multibeam RADAR sensors, use multiple transceivers to send out and receive a variety of radar beams simultaneously or in rapid succession. Single transceiver RADAR provides information about targets along a single line or sight at a given moment, while multibeam systems cover a wider field of view in the same period. In one embodiment, RADAR sensors include single transceiver RADAR sensors configured to operate in a dual-frequency RADAR mode with a single transceiver to emit and receive a variety of high-frequency and low-frequency RADAR pulses. Additionally, unlike multibeam RADAR sensors, which produce high-resolution, 3D image-like return signals that are computationally burdensome to process and analyze, single transceiver RADAR sensors produce lower resolution 2D images of the surface of the pavement that require less powerful computing resources to process and analyze.

The RADAR sensors may be configured to employ various frequencies, which may include multiple frequencies. In one example, RADAR sensors may utilize high-frequency and low-frequency RADAR pulses. Combinations of high-frequency and low-frequency pulses may be used to resolve different sized features (e.g. cracks, voids, and/or raveling) present in the surface of the pavement. To resolve the features, the RADAR sensors may emit both high-frequency and low-frequency RADAR pulses in rapid succession. Low-frequency RADAR pulses may include RADAR pulses with a frequency lower than 1 GHz. High-frequency RADAR pulses may include RADAR pulses with a frequency greater than 1 GHz. In some embodiments, high-frequency RADAR pulses may include RADAR pulses with a frequency between 2 GHz and 40 GHz. In some embodiments, the high-frequency RADAR pulses may be approximately double the frequency of the low-frequency RADAR pulses. For example, if the low-frequency RADAR pulses are emitted with a frequency of 500 MHZ, then the high-frequency RADAR pulses may be emitted with a frequency of 1 GHz.

The RADAR unit 124 may be configured to measure the power of the RADAR pulses emitted by the RADAR sensors and the power of the RADAR pulses reflected back at the RADAR unit 124. RADAR sensors may emit RADAR pulses directed at the surface of the pavement and detect reflected RADAR pulses produced as the emitted RADAR pulses interact with the surface of the pavement. The RADAR sensors may measure the power of the emitted RADAR pulse and the reflected RADAR pulses. The RADAR unit 124 may then generate sensor data, which may include the power of the emitted RADAR pulses and the power of the reflected pulses.

In some embodiments, the system 100 may implement a polarizing filter. A polarizing filter may polarize the RADAR pulses emitted by the RADAR sensors in different directions relative to cracks and/or voids in the surface of the pavement to determine the orientation of the cracks and/or voids. For example, if the RADAR pulse is polarized parallel to the length of the cracks and/or voids in the surface of the pavement, the RADAR pulse will interact more strongly with the cracks and/or voids, resulting in a weaker return signal. If the RADAR pulse is polarized perpendicular to the length of the cracks and/or voids in the surface of the pavement, the interaction between the RADAR pulse and the cracks and/or voids will be weaker, resulting in a stronger return signal. By comparing the magnitude of the return signal of differently polarized RADAR pulses, the orientation of the cracks and/or voids can be determined.

In one example, the polarizing filter may be implemented as part of the RADAR unit 124. In another example, the polarizing filter may be placed on or fixed on the exterior of the RADAR sensors.

In some embodiments, the camera unit 126 may include one or more cameras. The camera unit 126 may work in tandem with the GPS unit 128 to determine when to capture images of the pavement as the vehicle 110 is driven on the pavement. In some embodiments, the camera unit 126 may be preloaded with a set of “Pac Man Points”. “Pac Man Points” may be GPS coordinates that designate locations where the cameras will capture images of the pavement. For example, the “Pac Man Points” may be evenly spaced at 75 feet apart in both directions of all roads in the road network assigned to the vehicle 110.

The sensor platform 120 may include a plurality of sensor units 121 detachably mounted to a vehicle 110 as shown in FIG. 1A. In some embodiments, the system 100 may include a plurality of vehicles 110, each vehicle 110 equipped with a detachably mounted sensor platform 120. In other embodiments, the system 100 may include the plurality of sensor units 121 as a stand-alone system for collecting data as shown in FIG. 1B.

FIG. 1B illustrates an embodiment of system 100 where the sensor platform 120 may include a plurality of sensor units 121 and a computing device 130. The computing device 130 may include one or more processors 140, a memory 150, and one or more network interfaces 160. The memory 150 may include one or more tangible, non-transitory computer-readable media. The memory 150 may be configured to store data collected and transmitted by the sensor platform 120. The memory 150 may also be configured to store computer program instructions executable by the one or more processors 140 for operating the system 100. The network interfaces 160 may include any one or more wired and/or wireless network interface, including but not limited to Ethernet, WiFi, Bluetooth, or any other wired and/or wireless network interface now known or later developed that is suitable for enabling the computing device 130 to communicate with the system 100 and/or with one or more other computing devices via one or more networks.

In some embodiments, the computing device 130 may include one or more user interfaces include hardware such as a touch sensitive display, a computer keyboard, a mouse, or any other type of user interface now known or later developed that is suitable for providing a way for a computer user to interact with the computing device 130 and control one or more functions or operations of the system 100. In some embodiments, the computing device 130 is any of desktop computer, laptop computer, remote server, smart phone device, a tablet computer, or any other suitable type of computing device now known or later developed.

In some embodiments, the vehicle 110 may include an onboard computing system with a processor and a memory for temporarily receiving and storing sensor data collected by the sensor platform 120 before transmitting the collected sensor data to the computing device 130. The onboard processor may perform basic processing tasks.

In operation, the computing device 130 may be communicatively coupled to the system 100. In some examples, the system 100 shown in FIG. 2 is the same (or substantially the same) as the system 100 shown and described with reference to FIGS. 1A and 1B. The system 100 in FIG. 2 may include one or more processors 140, memory 150, and one or more network interfaces 160. The memory 150 may include one or more tangible, non-transitory computer-readable media. The memory 150 may be configured to store data collected and transmitted by the sensor platform 120. The memory 150 may also be configured to store computer program instructions executable by the one or more processors 140 for operating the system 100. The network interfaces 160 may include any one or more wired and/or wireless network interface, including but not limited to Ethernet, WiFi, Bluetooth, USB (e.g., USB-C) or any other wired and/or wireless network interface now known or later developed that is suitable for enabling the system 100 to communicate with one or more computing devices (e.g., computing device 130) via one or more networks.

FIG. 2 illustrates a processor 140 according to some embodiments. The processor 140 may include pavement condition generator 170. The pavement condition generator 170 may include a data processing unit 172, a sensor PCI estimation unit 174, and a pavement condition estimation unit 176. The pavement generator 170 may also include data storage for receiving sensor data transmitted by the sensor platform 120.

The data processing unit 172 may retrieve the sensor data from the memory 150, process the collected sensor data, and transmit the processed sensor data to the sensor PCI estimation unit 174. The sensor PCI estimation unit 174 may receive the processed sensor data from the data processing unit 172, determine a PCI value for each sensor unit of the sensor platform 120, based on the processed data, and transmit the determined PCI values to the pavement condition estimation unit 176.

The pavement condition estimation unit 176 may receive the determined PCI values for each sensor unit of the sensor platform 120 from the sensor PCI estimation unit 174, determine a fused PCI value based on the determined PCI values for each sensor unit of the sensor platform 120, assess pavement condition based on the determined fused PCI value, and output the determined fused PCI value, the assessed pavement condition, and an explanation of the correlation between the determined fused PCI value and the assessed pavement condition. The pavement condition estimation unit 176 may apply a sensor fusion algorithm to the determined PCI values for each sensor unit of the sensor platform 120 to determine the fused PCI value. The sensor fusion algorithm may include weighted values for each sensor unit of the sensor platform 120 that are applied to the determined PCI values for each sensor unit of the sensor platform 120. The weighted values may be based on historical data collected by that sensor unit of the sensor platform 120 during previous trip down the same paved road. The weighted values may also be based on metadata for each sensor unit of the sensor platform 120.

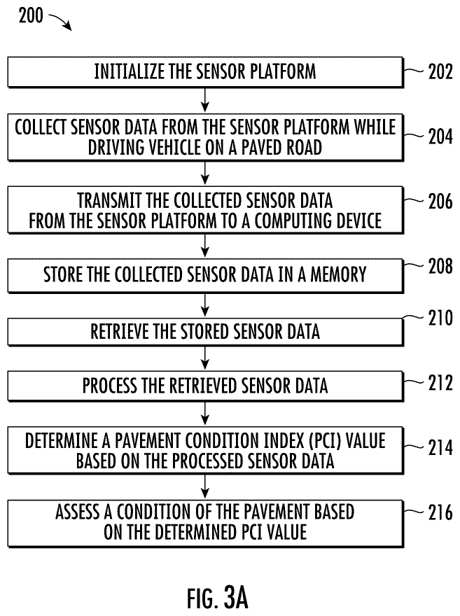

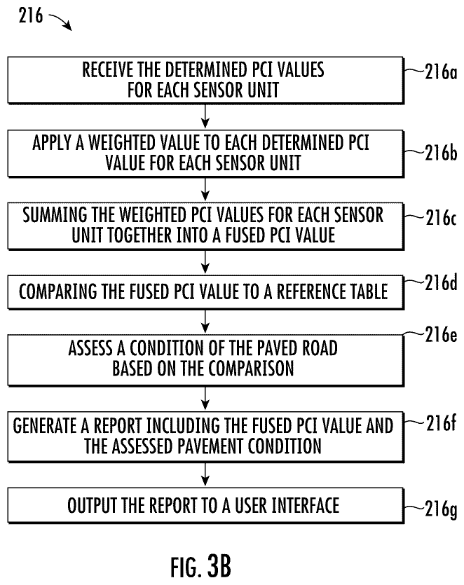

FIG. 3A illustrates a method 200 of assessing pavement according to some embodiments. For ease of explanation, certain aspects of method 200 are described with reference to the example system 100 and computing device 130 shown in FIGS. 1A and 1B. However, it will be appreciated that method 200 may be performed by systems or devices different than system 100. In some instances, the system 100 is used to assess the condition of pavement. In operation, one or more (or all) aspects of method 200 are performed by the system 100 individually or in combination with a separate computing device 130. In some embodiments where the system 100 may include one or more (or all) components of a computing device 130, the method 200 may be performed entirely by the system 100.

The method 200 may include initializing the sensor platform 202, collecting sensor data from the sensor platform while the vehicle is driving on a paved road 204, transmitting the collected sensor data from the sensor platform to the computing device 206, storing the collected sensor data in the memory 208, retrieving the stored sensor data from the memory 210, processing the retrieved sensor data 212, determining a pavement condition index (PCI) value based on the processed sensor data 214, and assessing a condition of the pavement based on the determined PCI value 216.

The method 200 may include initializing the sensor platform 202. In one example, with reference to system 100, the user may be prompted for an input to the initialize the sensor platform 120. Additionally or alternatively, the method 200 may include initializing the sensor platform autonomously without a user input, as described above and elsewhere herein.

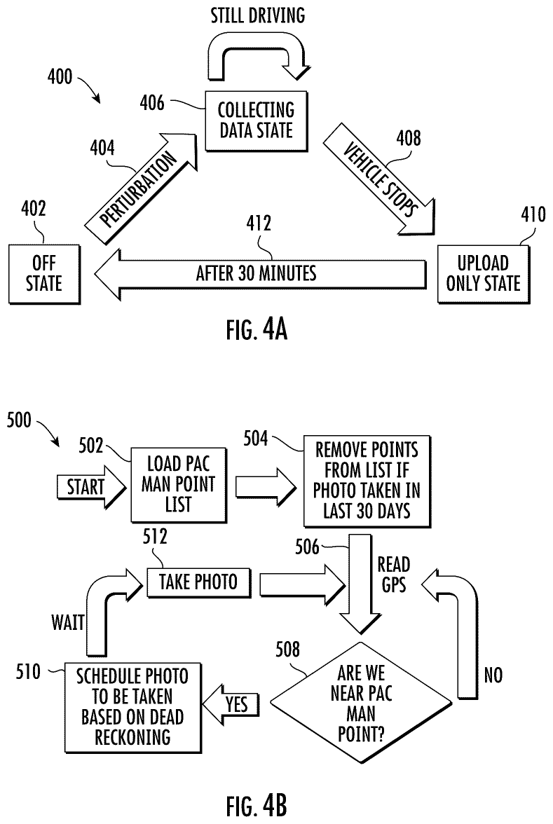

FIG. 4A illustrates a method 400 according to some embodiments of method 200 utilizing an accelerometer/IMU unit to autonomously initialize the sensor platform. The sensor platform may be initially in an “off-state” 402 where the majority of the sensor units may be powered down excepts for one or more of accelerometer/IMU sensors. For example, all the sensors of the sensor units may be powered down except an accelerometer/IMU. When one or more accelerometer/IMU sensors detect a significant perturbation 404 (like a door opening), the sensor platform may initialize the remaining sensor units and begin collecting sensor data 406. If one or more of accelerometer/IMU sensors detect that the vehicle has come to a stop 408 after initializing the sensor platform, the sensor platform may upload the collected sensor data to the memory 410. The sensor platform may also cause the sensors to stop collecting data. If after a predetermined time period 412 movement of the vehicle is not detected by the one or more of accelerometer/IMU sensors, the sensor platform may return to the “off-state” and power down sensors. Conversely, if the one or more of accelerometer/IMU sensors detect that the vehicle has moved or has been perturbed within the predetermined time period 412, the sensor platform may cause the sensors to again collect sensor data 406. In the illustrated embodiment, the predetermined time period is 30 minutes. However, the predetermined period of time may be set at longer or shorter periods of time, e.g., based on experience, available power, or as otherwise desired. In some embodiments, sensor platform 120 of system 100 may be configured to execute method 400. In some embodiments, the accelerometer/IMU signal used to autonomously initialize the sensor platform may not be processed in subsequent steps of method 200 discussed below.

With reference again to method 200 of FIG. 3A, the method 200 may collecting sensor data from each sensor unit of the sensor platform while the vehicle is driving on pavement.

As introduced above, the sensor platform of system 100 may be configured to collect sensor data with the sensor units while the vehicle is driving on a paved road 204, which may be a paved road within a road network assigned to the vehicle.

In one embodiment, the sensor data may include sensor data collected by the accelerometer/IMU unit. The accelerometer/IMU unit may include one or more accelerometer/IMU sensors that detect and measure changes in acceleration while the vehicle is driving on a paved road. The sensor data collected by the accelerometer/IMU unit may include accelerations detected and measured by the accelerometer/IMU sensors while the vehicle is driving on pavement. The accelerations may include the magnitude and the direction (the x-axis, y-axis, and/or z-axis) of the accelerations detected by the accelerometer/IMU sensors. The accelerometer/IMU unit may generate sensor data, which may include a time-series vector signal representing changes in 3D accelerations detected by accelerometer/IMU sensors over a predetermined time interval as the vehicle drives on the pavement.

In the above or another embodiment, the sensor data may include sensor data collected by the RADAR unit. The sensor data collected by the RADAR unit may include the power of RADAR pulses emitted by the RADAR sensors and the power of the RADAR pulses reflected back at the RADAR sensors. The RADAR sensors may emit RADAR pulses directed at the surface of the pavement while the vehicle is driving on pavement. The RADAR unit may then detect reflected RADAR pulses reflected back at the RADAR sensors as the emitted RADAR pulses interact with the surface of the pavement. The RADAR unit may then measure the power of the reflected RADAR pulses. The RADAR unit may then generate sensor data, which may include the power of the emitted RADAR pulses and the power of the reflected RADAR pulses.

In any of the above or another embodiment, the sensor data may include images captured by the camera unit. The images captured by the camera unit may include capturing images of the pavement using the one or more cameras.

FIG. 4B illustrates a method 500 according to some embodiments of method 200 utilizing a camera unit to capture images of the pavement at different locations as designated by the preloaded “Pac Man Points” while the vehicle is driving on a road. In one example, the road may be within a road network assigned to the vehicle, as previously discussed. The method 500 may include driving the vehicle on the road, monitoring the GPS position of vehicle using a GPS unit, and transmitting the GPS position to a computing device. In some embodiments, sensor platform 120 of system 100 may be configured to execute method 500.

The sensor platform may initially load the preloaded “Pac Man Point” list 502 from the cameras unit as previously discussed and receive the transmitted GPS location of the vehicle. Upon loading the “Pac Man Point” list, the sensor platform may add or remove “Pac Man Points” 504 from the preloaded “Pac Man Points” list based on whether an image was captured of those particular “Pac Man Point” within a previously elapsed time period. For example, the sensor platform may remove “Pac Man Points” from the preloaded “Pac Man Points” list if an image has been captured of those particular “Pac Man Points” within the last 30 days. If a “Pac Man Point” is removed from the preloaded “Pac Man Points” list, the sensor platform may add the previously removed “Pac Man Points” to the preloaded “Pac Man Points” list once 30 days has elapsed since the removal of the “Pac Man Points”. The sensor platform may read the current GPS position 506 of the vehicle from the GPS unit and compare the GPS position to the preloaded “Pac Man Points” list to determine if the vehicle is near a “Pac Man Point” 508. If the sensor platform determines that the vehicle is near a “Pac Man Point”, the sensor platform may schedule a photo to be taken of the pavement at that particular “Pac Man Point”. Once the vehicle arrives at the GPS location of the “Pac Man Point”, the sensor platform may generate a control signal that is transmitted to the camera unit, directing the one or more camera to capture an image of the pavement 512. The steps of method 500 are repeated until images are captured for all the preloaded “Pac Man Points” of the pavement within the assigned road network.

With reference again to FIG. 2, the method 200 may include transmitting the collected sensor data to the computing device.

In one example, the collected sensor data may be transmitted to a computing device 206, such as computing device 130. In one example, the collected sensor data may be further stored in memory of accessible by the computing device, such as memory 150.

In some embodiments, collecting sensor data from the sensor platform while traveling the paved road 204 and transmitting the collected senor data to the computing device 206 in method 200 may occur continuously. In one example, this collecting and transmitting may terminate upon receipt of a user input terminating the sequence. For example, system 100 may execute method 200 and the collecting and transmitting may terminate when the system 100 receives a user input terminating the sequence of collecting and transmitting sensor data.

In some embodiments, collecting sensor data from the sensor platform while traveling the paved road 204 and transmitting the collected senor data to the computing device 206, occur when the distance traveled by vehicle exceeds a predetermined distance threshold in a predetermined time period. For example, with respect to system 100, the GPS unit may track the distance driven by vehicle and the processor 140 may determine the distance driven by the vehicle. If the processor 140 determines that the distance driven by the vehicle falls under the predetermined distance in the predetermined time period, the system 100 is configured to attempt to transmit the sensor data collected during the predetermined time period to the memory 150. If after a predetermined period of time, the GPS unit and/or the accelerometer/IMU unit have not detected movement, the system 100 powers down terminating collection and transmission of sensor data.

The method 200 may include storing the collected sensor data in a memory 208 and retrieving the sensor data 210 stored in the memory.

In one example, with respect to the method 200 performed by system 100, storing the collected sensor data 208 may include transmitting the collected sensor data to a remote data storage device or the pavement condition generator. In some embodiments, storing the collected sensor data 208 may include transmitting the collected sensor data from the an onboard memory of the sensor platform to the pavement condition generator or a data storage device for storage until needed by the pavement condition generator. In some embodiments, the pavement condition generator may be in data communication with or otherwise configured to access the data storage device. The data storage device may be separate from, e.g., remote with respect to, the sensor platform. The pavement condition generator may reside on a server, which may include multiple servers, or other computing device or devices. The pavement condition generator may comprise a hardware-based server, software-based server, or both. The pavement condition generator, data storage device, or both may operate within a distributed computing environment. In one embodiment, the pavement condition generator, data storage device, or both may operate in a cloud based environment.

The stored data may be retrieved 210 for further processing. For example, system 100 may execute method 200 wherein the processing unit 172 may retrieve the sensor data stored in the memory 150 for further processing as described herein.

The method 200 may include processing the retrieved sensor data 212. For example, system 100 may execute method 200 wherein the processing unit 172 processes the retrieved sensor data. The data processing unit 172 may perform different processing techniques based on the type of sensor that collected that particular sensor data.

For example, processing the retrieved sensor data 212 may include processing the data collected from the accelerometer/IMU unit, using the data processing unit 172. Processing the sensor data collected from the accelerometer/IMU unit may include removing accelerations that are not in the direction of gravity. For example, the data processing unit 172 may remove any accelerations from the signal that are not in the direction of gravity, such as lateral or centripetal accelerations. The data processing unit 172 may remove these accelerations by converting the original time-series vector signal collected from the accelerometer/IMU unit into a scalar signal. The scalar signal may then subsequently be used in further processing by the sensor PCI estimation unit 174 to determine the PCI value for the sensor data collected from the accelerometer/IMU unit.

In another example, processing the retrieved sensor data 212 may include processing data collected by the RADAR unit, using the data processing unit 172. Processing the sensor data collected from the RADAR unit may include determining a RADAR peak power distance, RADAR peak power, and RADAR total power based on the sensor data collected by the RADAR sensors.

In some embodiments, the data processing unit 172 may resolve different sized features (e.g. cracks, voids, and/or raveling) present in the surface of the pavement by measuring and comparing the power of the reflected RADAR pulse for high frequency RADAR pulses and low frequency pulses. For example, if the data processing unit 172 determines that the power of the reflected RADAR pulse is maximized (e.g., the power of the reflected RADAR pulses is approximately the same as the power of the emitted RADAR pulses), then the surface of the pavement is smooth and relatively defect-free. If the data processing unit 172 determines that the power of the reflected RADAR pulse is low after emitting a high-frequency RADAR pulse, then either raveling or cracking may be present in the surface of the pavement. However, if the data processing unit 172 determines that there is no degradation in the power of the reflected RADAR pulse after emitting a low-frequency RADAR pulse at the same time as or immediately after the high-frequency RADAR pulse, then raveling is present, but cracking is not present because cracks would generate a drop in the power of the reflected RADAR pulse for the low-frequency RADAR pulse.

In another example, processing the retrieved sensor data 212 may include processing the images captured by the cameras unit, using the data processing unit 172. Processing the images captured by the camera unit may include converting the captured image from color space to another color space. For example, processing the captured images may include converting the captured images to the CIELAB color space and performing histogram equalization on the L (Lightness) channel on the converted sensor data. The equalized image may then be converted back to the RGB color space for further processing.

In some embodiments, processing the images captured by the camera unit may include detecting the pavement in the captured image using the data processing unit 172. The data processing unit 172 may utilize a convolution neural network (CNN) and the K-means clustering algorithm to detect the pavement in the captured image and exclude false positives in busy areas like trees, grass, and cars.

The CNN may be trained to perform binary classification on whether a subset of the captured image is part of the pavement. In one example, the CNN may be trained using a process called transfer learning. The model may be trained using a set of training images split into smaller “chunks”, each chunk labeled as pavement or non-pavement. The training image may then be input into the CNN. For classification purposes, the CNN may be trained to define pavement as the largest set of continuous, connected chunks predicted to be pavement. After the CNN is trained, the images captured by the camera unit may then be input into the CNN and the CNN may predict the portion of the captured images is pavement.

The K-means clustering algorithm may be used to find straight lines interpreted as the edge of the pavement in the images captured by the camera unit. A parallel K-Means implementation may be used to cluster each pixel to different colors. Yellow and White pixels may be representative of the yellow and white lines found on roads and green pixels may be representative of the grass/foliage bordering the pavement. These yellow, white, and green pixels may be passed to a Hough Line Transform algorithm to determine if the pixels are representative of straight lines in the captured images. The two lines most representative of the edge of the pavement may be chosen using metrics such as slope, length, and distance from the center of the captured image. The pavement may be defined as the area between the two chosen lines. If two suitable lines cannot be found in the captured image or the area between the two lines is sufficiently small, the captured image may be discarded, as there is not enough pavement to perform meaningful analysis.

The method 200 may include determining a PCI value for each sensor unit of the sensor platform based on the processed data 214.

In one example, determining a PCI value for each sensor unit of the sensor platform based on the processed data 214 may include determining the PCI value for the accelerometer/IMU unit as a function of three metrics using the sensor PCI estimation unit 174. The three metrics may include the MMD₈₀ value, the velocity of the scalar signal, and the Major Events Count.

The sensor PCI estimation unit 174 may determine the MMD₈₀ value by calculating a median-maximum discrepancy value using Equation 1 below:

$${MMD}_{80} = max ( middle\_ 80 \_ \% ( {S}_{filtered} ) ) - min ( middle\_ 80 \_ \% ( {S}_{filtered} ) ) \qquad Equation 1$$

In Equation 1, S_{filtered} represents the processed accelerometer/IMU signal for a paved road within the road network assigned to the vehicle from processing the retrieved sensor data 212. middle_80_% (S_{filtered}) represents the portion of the processed time-accelerometer/IMU signal that remains after removing the top 10% and bottom 10% of the processed accelerometer/IMU signal based on the spectral power distribution of the entire processed accelerometer/IMU signal.

The sensor PCI estimation unit 174 may determine the MMD₈₀ value by determining the maximum value for the middle 80% portion of the processed accelerometer/IMU signal, determining the minimum value for the middle 80% portion of the processed accelerometer/IMU signal, and subtracting the determined minimum value from the determined maximum value.

In some embodiments, the sensor PCI estimation unit 174 may normalize the MMD₈₀ value between vehicles in a fleet of vehicles. Normalizing the MMD₈₀ value may include calculating a multiplicative constant that is applied to the MMD₈₀ value of each vehicle before it is averaged. The sensor PCI estimation unit 174 may collect the sensor data from all the vehicles in the fleet for the 20 most driven sections of paved roads within the assigned road network and determine the MMD₈₀ value for each paved road and each vehicle. The sensor PCI estimation unit 174 may compile this data into feature vectors identifying each vehicle and each paved road. Each feature vector may include an ID number for the vehicle, the MMD₈₀ value for a particular paved road, the average velocity of the vehicle while it was driving on that particular paved road, and the road ID for the paved road. The sensor PCI estimation unit 174 may arbitrarily choose a road ID and a velocity range, then calculate the average MMD₈₀ value for all vehicles traveling within the velocity range on the paved road associated with the road ID. The sensor PCI estimation unit 174 may then determine a multiplicative constant for each vehicle such that the MMD₈₀ value for that vehicle is equal to the average MMD₈₀ value for the entire dataset. The sensor PCI estimation unit 174 may repeat the above steps 100 times to normalize the MMD₈₀ value for each vehicle in the fleet of vehicles.

The sensor PCI estimation unit 174 may determine the Major Events Count by using the Bollinger Bands statistical charting method to track the volatility of the scalar component of the accelerometer/IMU signal over time. Every time the moving average of the scalar component of the accelerometer/IMU signal exceeds a predetermined threshold of acceleration, each event is counted and recorded as part of the Major Event Count.

In some embodiments, the sensor PCI estimation unit 174 may determine the PCI value for the accelerometer/IMU unit based on accelerometer/IMU sensor data collected after the vehicle has completed a single trip on a road. In some embodiments, the sensor PCI estimation unit 174 may determine the PCI value for the accelerometer/IMU unit based on accelerometer/IMU sensor data collected after the vehicle has completed 40 trips on the same road. For example, the sensor PCI estimation unit 174 may determine a PCI value for the accelerometer/IMU unit for each trip of the 40 trips performed on the same road and average the PCI values together to produce a single PCI value for the accelerometer/IMU unit.

In another example, determining a PCI value for each sensor unit of the sensor platform based on the processed data 214 may include determining the PCI value for the RADAR unit as a function of the RADAR peak power distance, RADAR peak power, and RADAR total power from each RADAR sensor in the array of RADAR sensors using the sensor PCI estimation unit 174. In some embodiments, the PCI value for the RADAR unit may be determined as a ratio of the total power of the return RADAR signal to the peak power of the return RADAR signal.

In one example, if the surface of the paved road has few imperfections, then the surface of the paved road, then the emitted RADAR pulse will produce a negligibly small scattering effect as it interacts with the surface of the paved road, which is measured by the total power of the return RADAR signal. In this case, the total power of the return RADAR signal will be approximately equal to the peak power of the return RADAR signal, which results in a high PCI value. For example, the ratio of the total power of the return RADAR signal to the peak power of the return RADAR signal will be approximately equal to 1. In another example, if the surface of the paved road has imperfections such as cracks and raveling, then the emitted RADAR pulse will produce significant scattering as it interacts with the surface of the paved road, which is measured by the total power of the return RADAR signal. In this case, the total power of the return RADAR signal will be less than the peak power of the return RADAR signal, which results in a low PCI value. For example, the ratio of the total power of the return RADAR signal to the peak power of the return RADAR signal will be less than 1.

In yet another example, determining a PCI value for each sensor unit of the sensor platform based on the processed data 214 may include determining the PCI value for the cameras unit 16 as a function of crack density, crack length, and crack location on the image using the sensor PCI estimation unit 174. Cracks located on the bottom of the image are weighted more heavily in the classification as they are relatively clearer and larger due to their physical proximity to the camera unit.

In some embodiments, crack detection may be performed on all areas in the collected images marked as pavement by the data processing unit 172 and do not contain cars, yellow lines, or white lines. The image may be converted to grayscale. An image averaging process may then be performed on the portion of the collected images marked as pavement, which changes the value of each pixel to also be reflective of the pixels surrounding it. The averaged images may then be filtered before performing edge detection. Filtering may include applying a median filter and/or a bilateral filter. For example, the median filter, bilateral filter, and canny edge detection may be implemented by computer vision or machine learning algorithms, such as OpenCV. This process leads to many small false positive edges created due to the median and bilateral filters. Any set of edges which are sufficiently small may be deemed as noise and are removed. Detected cracks may be defined as the set of all edges remaining from canny edge detection after noise removal.

In some embodiments, the PCI value from the images captured by camera unit may be the average PCI values for each image in the last two months on a particular road with the road network assigned to the vehicle.

The method 200 may include assessing the condition of the pavement based on the determined PCI values for each sensor unit of the sensor platform 216 as determined at determining a PCI value for each sensor unit of the sensor platform based on the processed data 214.

FIG. 3B illustrates a flow chart of assessing the condition of the pavement based on the determined PCI values for each sensor unit of the sensor platform 216 according to some embodiments of method 200.

Assessing the condition of the pavement based on the determined PCI values for each sensor unit of the sensor platform 216 may include receiving the determined PCI values for each sensor unit of the sensor platform 216a. Receiving the determined PCI values for each sensor unit of the sensor platform 216a may include the pavement condition estimation unit 176 receiving the determined PCI values for each sensor unit of the sensor platform from the sensor PCI estimation unit 174.

Assessing the condition of the pavement based on the determined PCI values for each sensor unit of the sensor platform 216 may include applying a weighted value to each determined PCI value for each sensor unit of the sensor platform 216b. Applying a weighted value to each determined PCI value for each sensor unit of the sensor platform 216b may include using the pavement condition estimation unit 176 to determine a constant for each sensor unit based on metadata for each sensor unit. The metadata may be indicative of the quality of the sensor data collected by each sensor unit. The constants for each sensor unit may be determined by the pavement condition estimation unit 176 using Equation 2 below.

$$α + β + γ = 1 \qquad Equation 2$$

Equation 2 shows the relationship between the weighted values α, β, and γ.

The accelerometer/IMU measurement constant, a, may be determined as a function of the number of vehicles that have travelled down a particular road and the variance in the output of the scalar signal from the accelerometer/IMU unit. The accelerometer/IMU measurement constant may also be determined as a function of a number of trips of taken by a single vehicle down a particular road and the variance in the output of the scalar signal from the accelerometer/IMU unit. For example, the higher the variance of the output, the lower the reliability of the PCI estimate. Thus, high variance in PCI output from multiple trips or a low number of trips will decrease the accelerometer/IMU measurement constant. According to Equation 2, the sum of all the sensor coefficients should equal 1.

The camera measurement constant, γ, may be determined as a function of one or more of a sharpness estimate of the images taken by the camera unit and an estimate of the amount of actual road present in the images taken. For example, times of day with low incident sunlight or out of focus images will lower the camera measurement constant.

The RADAR measurement constant, β, may be the difference between 1−(α+Y).

Applying a weighted value to each determined PCI value for each sensor unit of the sensor platform 216b may further include multiplying the determined PCI value for each sensor unit of the sensor platform by the constant corresponding to that sensor unit using the pavement condition estimation unit 176.

Assessing the condition of the pavement based on the determined PCI values for each sensor unit of the sensor platform 216 may include summing the weighted PCI values for each sensor unit into a single fused PCI value 216c. Summing the weighted PCI values for each sensor unit into a single fused PCI value 216c may include adding the weighted PCI values for each sensor unit together into a single fused PCI value using the pavement condition estimation unit 176. The pavement condition estimation unit 176 may use a sensor fusion algorithm shown in Equation 3 below to calculate the fused PCI value.

$${PCI}_{Road} = α ( {PCI}_{Accelerometers} ) + β ( {PCI}_{Radar} ) + γ ( {PCI}_{Road Photos} ) \qquad Equation 3$$

Equation 3 shows the weighted values α, β, and γ applied to the PCI values previously determined in determining a PCI value for each sensor unit of the sensor platform based on the processed data 214 based on the sensor data from the accelerometer/IMU unit, RADAR unit, and the camera unit. The values of α, β, and γ may be determined by metadata. The metadata may indicate the quality of the data collected from each sensor unit of the sensor platform.

Assessing the condition of the pavement based on the determined PCI values for each sensor unit of the sensor platform 216 may include comparing the fused PCI value to a reference table 216d. Comparing the fused PCI value to a reference table 216d may include using the processor 140 or the pavement condition estimation unit 176 to compare the fused PCI value for the pavement to a reference table stored in the memory 150. In some embodiments, the reference table may include a list of PCI values and a description of the average pavement condition corresponding to each PCI value. In other embodiments, the reference table may include various ranges of PCI values and a description of the average pavement condition corresponding to each PCI range.

Assessing the condition of the pavement based on the determined PCI values for each sensor unit of the sensor platform 216 may include assessing a condition of the paved road based on the comparison 216e. Assessing a condition of the paved road based on the comparison 216e may include retrieving from the reference table the description of the average pavement condition corresponding to the PCI value or the range of PCI values closest to the fused PCI value based on the comparison between the fused PCI value and the reference table. The processor 140 or the pavement condition estimation unit 176 may retrieve the description of the average pavement condition from the reference table stored in the memory 150.

Assessing the condition of the pavement based on the determined PCI values for each sensor unit of the sensor platform 216 may include generating a report 216f and outing the report to a user interface 216g. Generating a report 216f may include compiling the fused PCI value, the description of the average pavement condition associated with the PCI value closest to the fused PCI value based on the comparison between the fused PCI value and the reference table, and an explanation of the correlation between the fused PCI value and the assessed pavement condition using the processor 140 or the pavement condition estimation unit 176.

Generating a report 216f may also include compiling additional statistics about the pavement condition based on the processed data from each sensor unit using the processor 140 or the pavement condition estimation unit 176. For example, the processor 140 or the pavement condition estimation unit 176 may compile statistics including the number of cracks detected on the pavement of a particular road in an assigned road network, the average length of detected cracks, the average width of detected cracks, the density of detected cracks (for example, number of detected cracks per square inch, per square foot, or per square mile), the amount of raveling detected on the pavement of a particular road in an assigned road network (reported as a percentage of raveling), and the severity of detected raveling.

Generating a report 216f may also include compiling a list of paved roads within the road network assigned to vehicle and ranking the paved roads based on pavement condition using the processor 140 or the pavement condition estimation unit 176. For example, the processor 140 or the pavement condition estimation unit 176 may rank the paved roads from worst condition to best condition, or vice versa.

The present disclosure may include dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example network or system is applicable to software, firmware, and hardware implementations.

In accordance with various embodiments of the present disclosure, the processes described herein may be intended for operation as software programs running on a computer processor. Furthermore, software implementations can include, but are not limited to, distributed processing or component/object distributed processing, parallel processing, or virtual machine processing that may be constructed to implement the methods described herein.

The present disclosure describes various systems, platforms, modules, units, devices, components, and the like. Such systems, platforms, modules, units, devices, components, and/or functionalities thereof may include one or more electronic processers, e.g., microprocessors, operable to execute instructions corresponding to the functionalities described herein. Such instructions may be stored on a computer readable medium. Such systems, platforms, modules, units, devices, components, and the like may include functionally related hardware, instructions, firmware, or software. For example, modules or units thereof, which may include generators or engines, may include physical or logical grouping of functionally related applications, services, resources, assets, systems, programs, databases, or the like. The systems, platforms, modules, units, which may include data storage devices such as databases and/or pattern library may include hardware storing instructions configured to execute disclosed functionalities, which may be physically located in one or more physical locations. For example, systems, platforms, modules, units, components or functionalities thereof may be distributed across one or more networks, systems, devices, or combination thereof. It will be appreciated that the various functionalities of these features may be modular, distributed, and/or integrated over one or more physical devices. It will be appreciated that such logical partitions may not correspond to physical partitions of the data. For example, all or portions of various systems, modules, units, or devices may reside or be distributed among one or more hardware locations.

The present disclosure contemplates a machine-readable medium containing instructions so that a device connected to the communications network, another network, or a combination thereof, can send or receive voice, video or data, and to communicate over the communications network, another network, or a combination thereof, using the instructions. The instructions may further be transmitted or received over the communications network, another network, or a combination thereof, via the network interface device. The term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that causes the machine to perform any one or more of the methodologies of the present disclosure. The terms “machine-readable medium,” “machine-readable device,” or “computer-readable device” shall accordingly be taken to include, but not be limited to: memory devices, solid-state memories such as a memory card or other package that houses one or more read-only (non-volatile) memories, random access memories, or other re-writable (volatile) memories; magneto-optical or optical medium such as a disk or tape; or other self-contained information archive or set of archives is considered a distribution medium equivalent to a tangible storage medium. The “machine-readable medium,” “machine-readable device,” or “computer-readable device” may be non-transitory, and, in certain embodiments, may not include a wave or signal per se. Accordingly, the disclosure is considered to include any one or more of a machine-readable medium or a distribution medium, as listed herein and including art-recognized equivalents and successor media, in which the software implementations herein are stored.

This specification has been written with reference to various non-limiting and non-exhaustive embodiments. However, it will be recognized by persons having ordinary skill in the art that various substitutions, modifications, or combinations of any of the disclosed embodiments (or portions thereof) may be made within the scope of this specification. Thus, it is contemplated and understood that this specification supports additional embodiments not expressly set forth in this specification. Such embodiments may be obtained, for example, by combining, modifying, or re-organizing any of the disclosed steps, components, elements, features, aspects, characteristics, limitations, and the like, of the various non-limiting and non-exhaustive embodiments described in this specification.

Various elements described herein have been described as alternatives or alternative combinations. It is to be appreciated that embodiments may include one, more, or all of any such elements. Thus, this description includes embodiments of all such elements independently and embodiments including such elements in all combinations.

The grammatical articles “one”, “a”, “an”, and “the”, as used in this specification, are intended to include “at least one” or “one or more”, unless otherwise indicated. Thus, the articles are used in this specification to refer to one or more than one (i.e., to “at least one”) of the grammatical objects of the article. By way of example, “a component” means one or more components, and thus, possibly, more than one component is contemplated and may be employed or used in an application of the described embodiments. Further, the use of a singular noun includes the plural, and the use of a plural noun includes the singular, unless the context of the usage requires otherwise.

What is claimed is:

1. A system for assessing pavement conditions comprising:
a sensor platform comprising one or more sensors units, wherein the sensor platform is configured to be coupled to a vehicle and collect sensor data with the one or more sensor units while the vehicle is driving on pavement, wherein one or more sensor units comprises a RADAR unit comprising one or more single-transceiver RADAR sensors;
a pavement condition generator configured to:
receive the collected sensor data from the sensor platform;
process the collected sensor data to remove outlier data;
determine a pavement condition index (PCI) value based on the processed sensor data; and
assess a condition of the pavement based on the determined PCI value.

2. The system of claim 1, wherein the one or more sensor units further comprises:
a GPS unit comprising one or more GPS modules;
an accelerometer unit comprising one or more accelerometers; and
a camera unit comprising one or more cameras.

3. The system of claim 1, wherein the RADAR unit emits high-frequency RADAR pulses and low-frequency RADAR pulses directed towards the pavement.

4. The system of claim 1, wherein further comprising a polarizing filter for polarizing RADAR pulses emitted by the RADAR unit in a direction either perpendicular or parallel to a surface of the pavement beneath the vehicle.

5. The system of claim 1, wherein the sensor platform is configured to simultaneously collect and upload the sensor data to a memory while the vehicle is driving on pavement.

6. The system of claim 2, wherein the pavement condition generator is further configured to determine a PCI value for each sensor unit based on the data collected by that sensor unit.

7. The system of claim 6, wherein the pavement condition generator is further configured to combine the determined PCI values for each sensor unit into a fused PCI value by applying a sensor fusion algorithm to the determined PCI values for each sensor unit.

8. The system of claim 7, wherein the sensor fusion algorithm applies a weighted value determined by metadata to each of the determined PCI values for each sensor unit,
wherein the metadata is indicative of a quality of the sensor data collected by each sensor unit.

9. The system of claim 2, wherein the pavement condition generator is further configured to:
monitor an accelerometer signal using the one or more accelerometers;
detect a perturbance in the accelerometer signal; and
initialize the sensor platform based on the detected perturbance.

10. The system of claim 2, wherein the pavement condition generator is further configured to:
monitor a distance traveled by the vehicle in a predetermined time period using the one or more GPS modules; and
initialize the sensor platform when the distance traveled by the vehicle exceeds a predetermined distance threshold within the predetermined time period.

11. A method of assessing pavement conditions comprising:
collecting sensor data from a sensor platform coupled to at least one vehicle while traveling over pavement, wherein the sensor platform comprises one or more sensor units, wherein one or more sensor units comprise at least a RADAR unit comprising one or more single-transceiver RADAR sensors;
processing the retrieved sensor data to remove anomalies from the retrieved data;
determining a pavement condition index (PCI) value based on the processed sensor data; and
assessing a condition of the pavement based on the determined PCI value.

12. The method of claim 11, wherein the one or more sensor units further comprise at least two sensor units selected from the group consisting of:
a GPS unit comprising one or more GPS modules;
an accelerometer unit comprising one or more accelerometers; and
a camera unit comprising one or more cameras.

13. The method of claim 11, wherein the RADAR unit emits high-frequency RADAR pulse and low-frequency RADAR pulses.

14. The method of claim 11, further comprising polarizing, using a polarizing filter, a RADAR pulse emitted by the RADAR unit in a direction either perpendicular or parallel to a surface of the pavement beneath the vehicle.

15. The method of claim 11, wherein the method further comprises simultaneously collecting, transmitting, and uploading the sensor data to a memory while the vehicle is driving on pavement.

16. The method of claim 12, wherein determining the PCI value based on the processed data further comprises determining a PCI value for each sensor unit based on the data collected by that sensor unit.

17. The method of claim 16, wherein assessing the condition of the pavement based on the determine PCI value further comprises combining the determined PCI value for each sensor unit into a fused PCI value by applying a sensor fusion algorithm to the determined PCI values for each sensor unit.

18. The method of claim 17, wherein the sensor fusion algorithm applies a weighted value determined by metadata to each of the determined PCI values for each sensor unit, wherein the metadata is indicative of a quality of the sensor data collected by each sensor unit.

19. The method of claim 11, wherein the method further comprises:
monitoring an accelerometer signal;
detecting a perturbance in the accelerometer signal; and
initializing the sensor platform based on the detected perturbance.

20. The method of claim 11, wherein the method further comprises:
monitoring a distance traveled by the vehicle in a predetermined time period using the GPS sensors; and
initializing the sensor platform when the distance traveled by the vehicle exceeds a predetermined distance threshold within the predetermined time period.
